(19) United States
(12) Patent Application Publication
(10) Pub. No.: US 2020/0086047 A1
(43) Pub. Date: Mar. 19, 2020

(54) PATIENT STIMULATION SYSTEM

(71) Applicant: Functional Neurosciences Inc., Toronto (CA)

(72) Inventors: Andres Lozano, Toronto (CA); J. Christopher Flaherty, Auburndale, FL (US)

(21) Appl. No.: 16/468,143

(22) PCT Filed: Dec. 14, 2017
(86) PCT No.: PCT/US2017/066339

§ 371 Date: Jun. 10, 2019

Related U.S. Application Data
(60) Provisional application No. 62/434,023, filed on Dec. 14, 2016.

(51) Int. Cl. A61M 5/172; A61B 5/055; A61B 5/0476; A61B 5/04; A61N 1/36; A61B 6/03; A61B 5/00

(52) U.S. Cl. CPC A61M 5/1723; A61B 5/055; A61B 5/0476; A61B 5/0042; A61N 1/36139; A61B 6/037; A61B 5/04001

(57) ABSTRACT

A stimulation system for treating a patient comprises: a stimulator comprising at least one stimulation element for delivering stimulation to the patient, the delivered stimulation based on a set of stimulation settings; a controller for adjusting a stimulation setting of the stimulator; and a diagnostic device for measuring a physiologic response to the test stimulation on the patient. The set of stimulation settings comprise a set of test stimulation settings and a set of therapeutic stimulation settings. The stimulation comprises test stimulation delivered to the patient for a test duration, and therapeutic stimulation delivered to the patient for an extended duration. The test stimulation is based on the set of test stimulation settings and the therapeutic stimulation is based on the set of therapeutic stimulation settings. The set of therapeutic stimulation settings is based on an assessment of the measured physiologic response. Methods of delivering stimulation are also provided.

### RELATED DOCUMENTS

The present application claims priority under 35 USC 119(e) to U.S. Provisional Application Ser. No. 62/434,023, entitled “Patient Stimulation System”, filed Dec. 14, 2016, which is hereby incorporated by reference in its entirety for all purposes.

### FIELD

The present invention relates generally to systems that provide therapeutic stimulation to a patient, such as systems that deliver therapeutic stimulation based on one or more measured patient parameters.

### BACKGROUND

Various patient diseases and disorders are treated with stimulation, such as electrical stimulation. For example, over 150,000 patients have received stimulation of the deep brain for the treatment of various neurological diseases and disorders including Parkinson's disease, depression, dystonia, tremor, pain, epilepsy, OCD and Alzheimer's disease.

Stimulators can be implanted within and/or external to the patient, and can deliver stimulation with adjustable stimulation settings. Determination of optimized or desired stimulation settings can be a complex process and it can require a long period of time. There is a need for stimulation systems that provide a simplified establishment of stimulation settings that provide improved therapy for the patient.

### SUMMARY

Embodiments of the systems, devices and methods described herein can be directed to systems, devices and methods for providing stimulation to one or more patient sites. The stimulation can provide delivery of energy such as electrical or other energy, and/or delivery of an agent such as a pharmaceutical agent.

According to an aspect of the present inventive concepts, a stimulation system for treating a patient comprises: a stimulator; a controller and a diagnostic device. The stimulator comprises at least one stimulation element for delivering stimulation to the patient, the delivered stimulation based on a set of stimulation settings. The set of stimulation settings comprise a set of test stimulation settings and a set of therapeutic stimulation settings. The stimulation comprises test stimulation delivered to the patient for a test duration, and therapeutic stimulation delivered to the patient for an extended duration. The test stimulation is based on the set of test stimulation settings and the therapeutic stimulation is based on the set of therapeutic stimulation settings. The controller is configured for adjusting a stimulation setting of the stimulator. The diagnostic device is configured for measuring a physiologic response to the test stimulation on the patient. For example, the diagnostic device can collect information related to one or more physiologic parameters of the patient, such that a physiologic response can be measured. The set of therapeutic stimulation settings is based on an assessment of the measured physiologic response.

In some embodiments, the system is configured to optimize therapeutic stimulation delivered by the stimulator. The system can be configured to perform the optimization by performing a function selected from the group consisting of: determine if the stimulator is delivering stimulation to a desired anatomical location; determine if the stimulator should deliver stimulation to a second anatomical location versus a first anatomical location; determine if the stimulator should deliver stimulation to multiple anatomical locations; determine if a selected anatomical location is achieving a desired effect; determine if the stimulator is causing an undesired effect to a non-target anatomical location; determine if the stimulator is delivering adequate stimulation; determine if the stimulator is delivering optimized stimulation; determine if the stimulator is delivering stimulation at an undesired level; and combinations thereof.

In some embodiments, the system is configured to manually assess the set of test stimulation settings.

In some embodiments, the system is configured to semi-automatically assess the set of test stimulation settings.

In some embodiments, the system is configured to automatically assess the set of test stimulation settings.

In some embodiments, the system is configured to allow an operator to manually adjust at least one test stimulation setting.

In some embodiments, the system is configured to semi-automatically adjust at least one test stimulation setting. The stimulation system can be configured to perform the semi-automatic adjustment after receipt of confirmation by a user.

In some embodiments, the system is configured to automatically adjust at least one test stimulation setting. The stimulation system can be configured to perform the automatic adjustment after receipt of confirmation by a user.

In some embodiments, the system is configured to treat one or more diseases and/or disorders selected from the group consisting of: a neurological disease; a neurological disorder; a psychiatric condition; Alzheimer's Disease (AD) such as Mild or Moderate Alzheimer's Disease; probable Alzheimer's Disease; a genetic form of Alzheimer's Disease; Mild Cognitive Impairment (MCI); hippocampal damage such as hippocampal damage due to Alzheimer's Disease, anoxia, epilepsy or depression; neuronal loss; neuronal damage; chemotherapy induced memory impairment; epilepsy; a seizure disorder; a movement disorder; essential tremor; dementia; amnesia; a memory disorder such a spatial memory disorder; head injury; traumatic brain injury; stroke; cognitive impairment associated with Schizophrenia; Parkinson's Disease related cognitive impairment or dementia; Parkinson-Plus syndrome; depression such as major depression; obsessive compulsive disorder (OCD); dystonia; addiction; cluster headache; anorexia nervosa; bipolar disorder; pain such as chronic pain; dementia such as dementia with Lewy bodies (DLB); Huntington's disease; obesity; Tourette syndrome; multiple sclerosis (MS) tremor; urinary function; and combinations thereof.

In some embodiments, the system is configured to treat two or more diseases and/or disorders selected from the group consisting of: a neurological disease; a neurological disorder; a psychiatric condition; Alzheimer's Disease (AD) such as Mild or Moderate Alzheimer's Disease; probable Alzheimer's Disease; a genetic form of Alzheimer's Disease; Mild Cognitive Impairment (MCI); hippocampal damage such as hippocampal damage due to Alzheimer's Disease, anoxia, epilepsy or depression; neuronal loss; neuronal damage; chemotherapy induced memory impairment; epilepsy; a seizure disorder; a movement disorder; essential tremor; dementia; amnesia; a memory disorder such a spatial memory disorder; head injury; traumatic brain injury; stroke; cognitive impairment associated with Schizophrenia; Parkinson's Disease related cognitive impairment or dementia; Parkinson-Plus syndrome; depression such as major depression; obsessive compulsive disorder (OCD); dystonia; addiction; cluster headache; anorexia nervosa; bipolar disorder; pain such as chronic pain; dementia such as dementia with Lewy bodies (DLB); Huntington's disease; obesity; Tourette syndrome; multiple sclerosis (MS) tremor; urinary function; and combinations thereof.

In some embodiments, the system is configured to adjust the stimulation settings in a closed loop fashion. The diagnostic device can comprise an implantable diagnostic device. The system can be configured to adjust the stimulation settings relatively continuously. The system can be configured to adjust the stimulation settings on a timeframe selected from the group consisting of: at least once a day; at least once a week; at least once a month; at least once every 3 months; and combinations thereof.

In some embodiments, at least a portion of the stimulator is implantable.

In some embodiments, the stimulator comprises an externally-placed stimulator.

In some embodiments, the stimulator comprises a housing configured for implantation into the patient. The housing can surround at least a portion of the diagnostic device.

In some embodiments, the at least one stimulation element is configured to deliver an agent to the patient. The at least one stimulation element can be further configured to deliver energy to the patient.

In some embodiments, the at least one stimulation element is configured to deliver energy to the patient. The delivered energy can comprise a form of energy selected from the group consisting of: electrical energy; microwave energy; magnetic energy; sound energy such as ultrasound energy; light energy; thermal energy; heat energy; cryogenic energy; chemical energy; and combinations thereof.

In some embodiments, the at least one stimulation element comprises a component selected from the group consisting of: electrode; optical component; piezo material; drug delivery element; and combinations thereof.

In some embodiments, the at least one stimulation element is configured to be positioned proximate a target location to be stimulated.

In some embodiments, the at least one stimulation element is configured to be positioned proximate a target location to be stimulated and a non-target location comprising a location to which no or minimal stimulation is to be delivered.

In some embodiments, the at least one stimulation element is configured to be positioned at a location selected from the group consisting of: a location on the skin; a skin location proximate an organ; a skin location proximate the brain; a skin location proximate the spine; an implant location; a location proximate the brain; a location proximate the vagus nerve; a location proximate the spine; a location proximate the heart; a location proximate the kidney; a location proximate the stomach; and combinations thereof.

In some embodiments, the at least one stimulation element is configured to be positioned at a location selected from the group consisting of: brain tissue; deep tissue of the deep brain; cortical brain tissue; nerve tissue; vagus nerve tissue; organ tissue; heart tissue; kidney tissue; pancreatic tissue; spinal cord tissue; central nervous system tissue; peripheral nervous system tissue; dorsal root ganglia; and combinations thereof.

In some embodiments, the at least one stimulation element is configured to be positioned at a brain location selected from the group consisting of: Papez Circuit; hippocampus; cingulate gyrus; fornix; a mammillothalamic tract; amygdala; hypothalamus; mammillary bodies; septal nuclei; temporal neocortex; the medial forebrain bundle (MFB); anterior and mediodorsal nuclei of the thalamus; the diagonal band of the Broca; temporal stem and temporal white matter; brainstem; nucleus basalis of Meynert; anterior thalamic nucleus; entorhinal cortex; rhinal cortex; periventricular zone; anterior thalamus; anterior insula; caudate; dorsal anterior cortex; dorsal cingulate; medial frontal cortex; nucleus accumbens; orbital frontal cortex; parietal region; periaqueductal gray area; posterior cingulate area; subcallosal area; subcallosal cingulate; subgenual cingulate; Brodmann area 10; Brodmann area 24; Brodmann area 25; Brodmann area 11/Brodmann area 10; Brodmann area 24b; Brodmann area 31; Brodmann area 32/Brodmann area 10; Brodmann area 32/Brodmann area 11; Brodmann area 39; Brodmann area 46; Brodmann area 46/Brodmann area 9; Brodmann area 47; Brodmann area 6; Brodmann area 9; one or more of Brodmann areas 1 through 52; ventral/medial prefrontal cortex area; ventral/medial white matter; dorsolateral prefrontal cortex; premotor cortex; ventrolateral prefrontal cortex; dorsal anterior cingulate caudate nucleus; frontal pole periaqueductal gray area; dorsolateral prefrontal area; subsingular cingulate; parahippocampal cortex; parahippocampal gyrus; ventral capsule; ventral striatum; ventral intermediate nucleus (VIM); ventral oralis posterior nucleus (VOP); ventral oralis anterior nucleus (VOA); posterior ventral globus pallidus internal; anterior medial globus pallidus internal; globus pallidis external; subthalamic nucleus; putamen; centromedian nucleus; zona incerta; preleminscal radiation; pedunculopontine nucleus; ventral posteromedial nucleus; ventral posterolateral nucleus; superior cingulum; septal area; ventral oral internal thalamic nuclei; dorsal medial nucleus; inferior thalamic peduncle; bed nucleus of the stria terminalis; stria terminalis; subcallosal cingulate gyrus CG25; latterly rostral anterior cingulate cortex CG24; lateral habenula; area LC of the caudate nucleus; anterior medial hypothalamus; posterior medial hypothalamus; lateral hypothalamus; orbito-frontal area; and combinations thereof.

In some embodiments, the at least one stimulation element comprises a sensor. The sensor can comprise a sensor configured to record electrical activity of tissue proximate the sensor.

In some embodiments, the set of stimulation settings comprise one or more stimulation settings.

In some embodiments, the stimulation setting comprises one or more settings selected from the group consisting of: anatomical position of the at least one stimulation element, anatomical position of a lead comprising the at least one stimulation element, selection of a subset of two or more stimulation elements; amplitude of energy delivery; frequency of stimulation delivered; pulse width of energy delivery; duration of stimulation delivery; time period in which stimulation is not to be delivered; flow rate of delivery of an agent; concentration of an agent being delivered; pressure at which an agent is delivered; duration of stimulation; and combinations thereof.

In some embodiments, the stimulation settings comprise a position of the at least one stimulation element. The at least one stimulation element can be adjusted based on the measured physiologic response. The stimulator can further comprise a lead onto which the at least one stimulation element is positioned, and the adjustment can comprise repositioning the lead. The at least one stimulation element can comprise multiple stimulation elements, and the adjustment can comprise changing stimulation elements delivering stimulation.

In some embodiments, the test duration comprises a time period of at least 1 second, at least 2 seconds, at least 3 seconds, at least 5 seconds, at least 10 seconds, at least 20 seconds, at least 1 minute, at least 5 minutes, at least 15 minutes, and/or at least 1 hour.

In some embodiments, the test duration comprises a time period of no more than 5 minutes, no more than 15 minutes, no more than 1 hour, and/or no more than 2 hours. In these embodiments, the diagnostic device can comprise an MRI and/or an electroencephalograph.

In some embodiments, the test duration comprises a time period of no more than 1 day, no more than 1 week, no more than 1 month and/or no more than 3 months.

In some embodiments, the test duration comprises a time period of approximately 5 seconds, approximately 10 seconds, approximately 20 seconds, approximately 1 minute, approximately 5 minutes, approximately 15 minutes, and/or approximately 1 hour.

In some embodiments, the extended duration comprises a period of time selected from the group consisting of: at least 1 week; at least 1 month; at least 6 months; and/or at least 1 year.

In some embodiments, the controller comprises a processing unit.

In some embodiments, the system further comprises an algorithm. The algorithm can be configured to assess one or more physiologic parameters (e.g. physiologic parameter data collected by the diagnostic device), such as to measure the physiologic response. The algorithm can be configured to assess a change in a physiologic parameter. The algorithm can be configured to assess a physiologic response, (e.g. by analyzing physiologic data obtained by the diagnostic device). The system can be configured to deliver energy and to delivery an agent, and the algorithm can be configured to optimize at least one of the energy delivery or the agent delivery. The algorithm can be configured to optimize the energy delivery and the agent delivery. The algorithm can be configured to perform at least two assessments. The algorithm can be configured to assess therapeutic benefit and presence of an undesired event. The algorithm can be configured to perform a comparison of two or more of: delivery of stimulation to a first location; delivery of stimulation to a second location; and/or delivery of stimulation to both the first location and the second location. The algorithm can comprise a biased algorithm (e.g. an algorithm with one or more biases). The algorithm can be biased to avoid an undesired event, and/or the algorithm can be configured to optimize a therapeutic benefit. The algorithm can be configured to select between two sets of stimulation settings that achieve similar therapeutic benefit, and the selection can be based on the avoidance of the undesired event. The algorithm can comprise a bias related to a non-therapeutic parameter. The non-therapeutic parameter can comprise battery life of the stimulator. The algorithm can be configured to select between two sets of stimulation settings that achieve similar therapeutic benefit, and the selection can be based on increasing battery life of the stimulator. The algorithm can comprise a bias related to: desired versus undesired implantation locations of the at least one stimulation element and/or other portion of the stimulator; desired versus undesired system configurations; and/or desired versus undesired agent to be delivered.

In some embodiments, the controller comprises electronic memory circuitry. The system can store a desired signature of neural activity in the electronic memory circuitry, and the set of therapeutic stimulation settings can be based on a comparison to the stored desired signature. The system can store a desired level of at least one physiologic parameter, and the set of therapeutic stimulation settings can be based on a comparison to the stored desired level. The system can store a desired level of a physiologic response, and the set of therapeutic stimulation settings can be based on a comparison to the stored desired level.

In some embodiments, the diagnostic device measures at least one physiologic parameter of the patient, and the physiologic response is based on the measurement of the at least one physiologic parameter. The at least one physiologic parameter can comprise at least one surrogate physiologic parameter representative of achievement of therapeutic benefit, and at least one surrogate physiologic parameter representative of occurrence of an undesired event.

In some embodiments, the diagnostic device comprises a device selected from the group consisting of: magnetic resonance imaging (MRI) such as functional magnetic resonance imaging (fMRI); electroencephalograph (EEG); magnetoencephalograph (MEG); positron emission tomography (PET) scanner; local field potential (LFP) recording device; neuronal spike recording device; MR spectroscopy device; MR angiography device; a regional blood flow measurement device (e.g. a device using perfusion CT and/or stable xenon CT); and combinations thereof.

In some embodiments, the diagnostic device comprises two or more devices selected from the group consisting of: magnetic resonance imaging (MRI) such as functional magnetic resonance imaging (fMRI); electroencephalograph (EEG); magnetoencephalograph (MEG); positron emission tomography (PET) scanner; local field potential (LFP) recording device; neuronal spike recording device; MR spectroscopy device; MR angiography device; a regional blood flow measurement device (e.g. a device using perfusion CT and/or stable xenon CT); and combinations thereof.

In some embodiments, the physiologic response comprises a change in a physiologic parameter. The change in a physiologic parameter can comprise a change as compared to a baseline condition in which no stimulation is delivered. The physiologic parameter can comprise a parameter selected from the group consisting of: neuronal firing activity; neuronal firing rate; power of brain activity; rhythm of brain activity; phase-amplitude coupling of brain activity; cerebral vascular transit time; glucose handling; and combinations thereof.

In some embodiments, the physiologic response comprises a change in neuronal activity. The physiologic response can comprise a change in brain neuronal activity.

In some embodiments, the measured physiologic response comprises a response measured after stimulation is applied for a minimum time duration.

In some embodiments, the system is configured to measure a first physiologic parameter after a first test stimulation is delivered and to measure a second physiologic parameter after a second test stimulation is delivered, and the first test stimulation can be based on a first set of test stimulation settings and the second test stimulation is based on a second set of test stimulation settings. The second test stimulation is delivered after a waiting duration has elapsed since the delivery of the first test stimulation. The waiting duration can comprise a time period of at least 10 seconds, at least 30 seconds, at least 1 minute, at least 15 minutes, and/or at least 1 hour. The waiting duration can comprise a time period of no more than 1 minute, no more than 5 minutes, no more than 30 minutes, no more than 1 hour and/or no more than 4 hours.

In some embodiments, the system further comprises a sensor configured to measure a physiologic parameter of the patient. The therapeutic stimulation settings can be based on an assessment of the sensor measurement. The stimulator can comprise the sensor. The stimulator can comprise at least a portion of the diagnostic device. The stimulator can comprise an implantable stimulator. The diagnostic device can comprise the sensor. The sensor can comprise an implantable sensor. The system can be configured to adjust the stimulation settings based on the sensor measurement and in a closed loop fashion. The stimulation settings can be adjusted on a relatively continuous basis. The stimulation settings can be adjusted at a time period of: at least once a day; at least once a week; at least once a month; and/or at least once every 3 months. The sensor can comprise an implantable sensor. The sensor can measure electrical activity. The sensor can measure a physiologic parameter selected from the group consisting of: neuronal spikes; LFP activity; EEG activity; and combinations thereof. The sensor can measure a physiologic parameter selected from the group consisting of: a brain chemical; a neurotransmitter; a protein; pH; glucose; and combinations thereof. The sensor can comprise one or more sensors selected from the group consisting of: an electrical activity sensor; an electrode-based stimulation element; a chemical sensor; an electromagnetic sensor; a pressure sensor; a temperature sensor; a neurotransmitter sensor; a protein sensor; a pH sensor, a glucose sensor; and combinations thereof. The sensor can comprise at least two sensors. The therapeutic stimulation settings can be based on measurements performed by the at least two sensors. The at least one stimulation element can comprise the sensor.

According to another aspect of the present inventive concepts, a method of delivering stimulation treatment to a patient comprises: (a) selecting a patient to receive stimulation therapy; (b) setting a first set of test stimulation settings; (c) delivering test stimulation to the patient for a test duration, the test stimulation comprising the first set of test stimulation settings; (d) measuring the physiologic response of the test stimulation on the patient; (e) assessing the physiologic response; (f) adjusting at least one test stimulation setting based on an undesired result from the assessment of step (e); (g) repeating steps (d) through (f) until a desired result is achieved; (h) setting a set of treatment stimulation settings based on the set of test stimulation settings that achieved the desired result; and (i) delivering treatment stimulation based on the treatment stimulation settings.

In some embodiments, the method further comprises gathering baseline data by measuring a physiologic parameter with no stimulation applied, and the physiologic response assessed in step (e) comprises comparison of a physiologic parameter with stimulation applied versus the same physiologic parameter without stimulation applied.

In some embodiments, step (f) includes adjusting a set of stimulation elements that deliver stimulation to the patient. A subsequent or prior performance of step (f) comprises adjusting a different stimulation setting.

In some embodiments, step (f) comprises adjusting a stimulation setting selected from the group consisting of: intensity such as voltage, current, amplitude, and/or magnetic field strength; frequency such as variation of one or more frequencies of a signal; pulse width; flow rate of an agent being delivered; concentration of an agent being delivered; pressure at which an agent is delivered; and combinations thereof.

In some embodiments, step (f) comprises adjusting a stimulation setting selected from the group consisting of: frequency of one or more waveforms of the stimulation such as a frequency variation between 1 Hz and 10,000 Hz; pulse width of one or more pulses of one or more waveforms of the stimulation such as a pulse width variation between 1 μsec and 500 μsec; intensity of one or more portions of one or more waveforms of the stimulation such as a peak or average current variation between 0.1 mA and 10 mA and/or a peak or average voltage variation between 1.0V and 10.0V; a theta burst property such as on or off; stimulation pattern such as a variation in stimulation waveform shape; and combinations thereof.

In some embodiments, the adjusting of the stimulation setting performed in step (f) is configured to increase activity in a first tissue area.

In some embodiments, the adjusting of the stimulation setting performed in step (f) is configured to increase activity in a first tissue area and decrease activity in a second tissue area.

In some embodiments, steps (c) thru (g) are repeated after the performance of step (h). Steps (c) thru (g) can be repeated after a minimum time duration selected from the group consisting of: 1 week; 1 month; 6 months; and 1 year.

The technology described herein, along with the attributes and attendant advantages thereof, will best be appreciated and understood in view of the following detailed description taken in conjunction with the accompanying drawings in which representative embodiments are described by way of example.

### BRIEF DESCRIPTION OF THE DRAWINGS

FIG. 1 illustrates a schematic view of a system for delivering stimulation to a patient, consistent with the present inventive concepts.

FIG. 1a illustrates a schematic view of a system for delivering stimulation to a patient including a diagnostic device internal to a stimulator, consistent with the present inventive concepts.

FIG. 2 illustrates a flow chart of a method for delivering stimulation to a patient, consistent with the present inventive concepts.

FIG. 3 illustrates a schematic anatomical view of a stimulation portion of a stimulation device implanted into a patient, consistent with the present inventive concepts.

FIG. 4 illustrates fMRI data collected during human clinical studies performed by applicant.

### DETAILED DESCRIPTION OF THE DRAWINGS

Reference will now be made in detail to the present embodiments of the technology, examples of which are illustrated in the accompanying drawings. The same reference numbers are used throughout the drawings to refer to the same or like parts.

It will be understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.).

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of one or more of these.

As used herein, the terms “about” or “approximately” shall refer to ±10%.

As used herein, the term “proximate” shall include locations relatively close to, on, in and/or within a referenced component, anatomical location and/or other location.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence.

The term “and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

The term “transducer” where used herein is to be taken to include any component or combination of components that receives energy or any input, and produces an output. For example, a transducer can include an electrode that receives electrical energy, and distributes the electrical energy to tissue (e.g. a distribution based on the size of the electrode). In some configurations, a transducer converts an electrical signal into any output, such as light (e.g. a transducer comprising a light emitting diode or light bulb), sound (e.g. a transducer comprising a piezo crystal configured to deliver ultrasound energy), pressure, heat energy, cryogenic energy, chemical energy; mechanical energy (e.g. a transducer comprising a motor or a solenoid), magnetic energy, and/or a different electrical signal (e.g. a Bluetooth or other wireless communication element). Alternatively or additionally, a transducer can convert a physical quantity (e.g. variations in a physical quantity) into an electrical signal. A transducer can include any component that delivers energy and/or an agent to tissue, such as a transducer configured to deliver one or more of: electrical energy to tissue (e.g. a transducer comprising one or more electrodes); light energy to tissue (e.g. a transducer comprising a laser, light emitting diode and/or optical component such as a lens or prism); mechanical energy to tissue (e.g. a transducer comprising a tissue manipulating element); sound energy to tissue (e.g. a transducer comprising a piezo crystal); chemical energy; electromagnetic energy; magnetic energy; and combinations of one or more of these.

As used herein, the term “desired” is to be taken to define an optimized, sufficient (e.g. clinically sufficient) or otherwise desired level of a parameter, condition or outcome, such as a level of a physiologic parameter or a level of a stimulation setting that correlates to an optimized, beneficial or otherwise sufficient therapy for the patient. A desired level of a parameter shall include any desired state of that parameter, such as a desired amplitude, frequency, pulse width or other condition. In some embodiments, a desired level of a parameter comprises a desired relationship between two or more parameters (e.g. a desired quantitative ratio or other relationship measurable by a diagnostic device of the present inventive concepts).

As used herein, the term “optimize” is to be taken to include optimizing or at least improving a condition from a previous state. One or more stimulation settings of the present inventive concepts can be optimized to improve a therapeutic result versus a result that would be achieved with a different set of stimulation settings. Alternatively or additionally, one or more stimulation settings can be optimized to reduce an adverse event or other undesired event (e.g. presence of any undesired condition during or otherwise as a result of stimulation delivery).

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination. For example, it will be appreciated that all features set out in any of the claims (whether independent or dependent) can be combined in any given way.

Provided herein are systems, devices and methods for delivering stimulation to a patient to treat one or more diseases or disorders. Stimulation elements of the system can be positioned on, within and/or otherwise proximate (singly or collectively “proximate” herein) one or more anatomical locations of the patient. Test stimulation, comprising stimulation based on a set of test stimulation settings, is delivered by a stimulator for a test duration (e.g. a relatively short period of time), and a diagnostic device is used to measure a physiologic response of the patient to the test stimulation (e.g. collect physiologic data of the patient after stimulation is delivered for a target or minimum time period, the collected physiologic data used by system 10 to determine the physiologic response). A measured physiologic response can comprise a change in one or more physiologic parameters of the patient, such as a change in brain or other neuronal activity. An assessment of the measured physiologic response is performed, to determine if the physiologic response is at a desired level (e.g. exceeds a threshold as described herein). If not, one or more test stimulation settings can be adjusted (e.g. manually, semi-automatically and/or automatically) and the modified test stimulation delivered (for a similar or dissimilar test duration), and a new measurement of the physiologic response performed. These steps can be repeated until a desired physiologic response is achieved, after which therapeutic stimulation can be delivered based on the final (acceptable) set of stimulation settings.

In some instances, choosing a desired set of stimulation settings can be relatively straight forward, such as when there is an immediate change in a symptom or behavior coincident with the application of the stimulation (e.g. electrical stimulation of the brain or the spine). In some pain applications (e.g. back pain), stimulation of nerves or other tissue can result in immediate or near-immediate pain relief. In patients with tremor for example, thalamic stimulation of the deep brain can cause an immediate cessation of tremor. However, in other disorders such as Parkinson's disease, depression, dystonia, epilepsy and Alzheimer's disease, there is little or no immediate clinical effects of the stimulation. The clinical benefits are often delayed, changes can be progressive over time, and one can never be certain a priori that the provided stimulation settings are optimized to produce a desired clinical benefit and/or reduce possible adverse effects. For these and other reasons, there is an urgent need to develop surrogate markers (a biomarker and/or other measurable surrogate physiologic parameter, “physiologic parameters” herein) and methods of optimizing the selection of stimulation settings that are not dependent on an immediate observable change in behavior or amelioration in symptoms. The systems, devices and methods of the present inventive concepts include utilizing such physiologic parameters and adjusting the stimulation delivered to achieve desired levels and/or desired changes (“desired change” herein) in those biomarkers. With deep brain stimulation, the stimulation system of the present inventive concepts can treat a malfunction within brain circuits, and the effect of the stimulation delivered can cause characteristic changes in the behavior and activity of those brain circuits. The various diagnostic devices described herein can be used to measure the resting activity of these brain circuits. The present inventive concepts include examining the baseline state of pathological brain circuits in psychiatric and/or neurologic disorders, and applying and adjusting stimulation (e.g. in a manual, semi-automatic and/or automatic closed looped arrangement) until a desired change in the activity of the circuit (as measured by one of these diagnostic devices) is achieved. In some embodiments, a baseline measure collected using EEG, PET, fMRI, MEG, and/or other diagnostic device as described herein is obtained. The stimulation is applied and the measurement by the diagnostic device is repeated. A determination of whether the desired physiological response has been achieved is made. If an undesired result is determined, the stimulation is accordingly adjusted (e.g. up or down) in an iterative way, with repeated diagnostic measurements performed until a desired physiologic response is achieved.

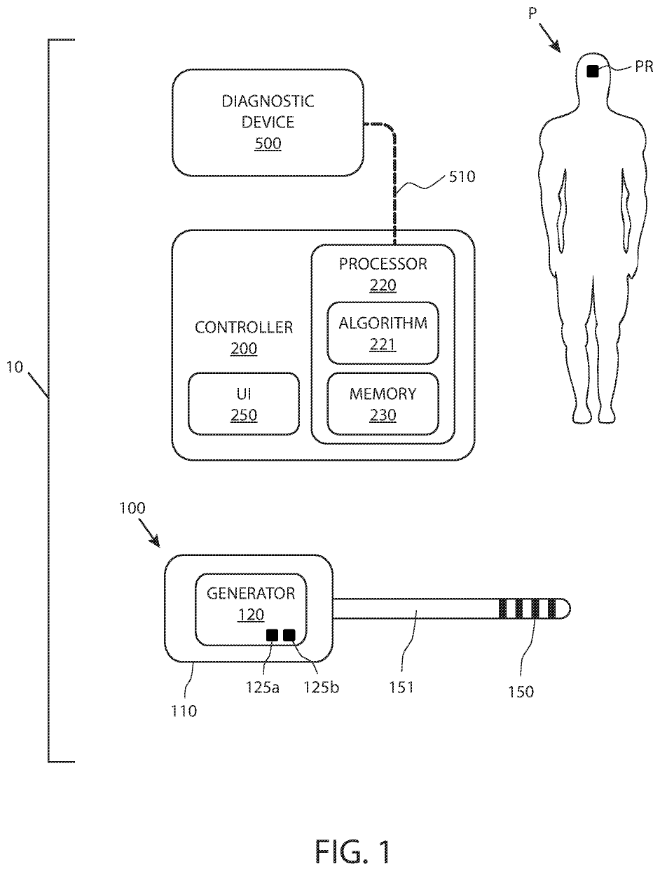

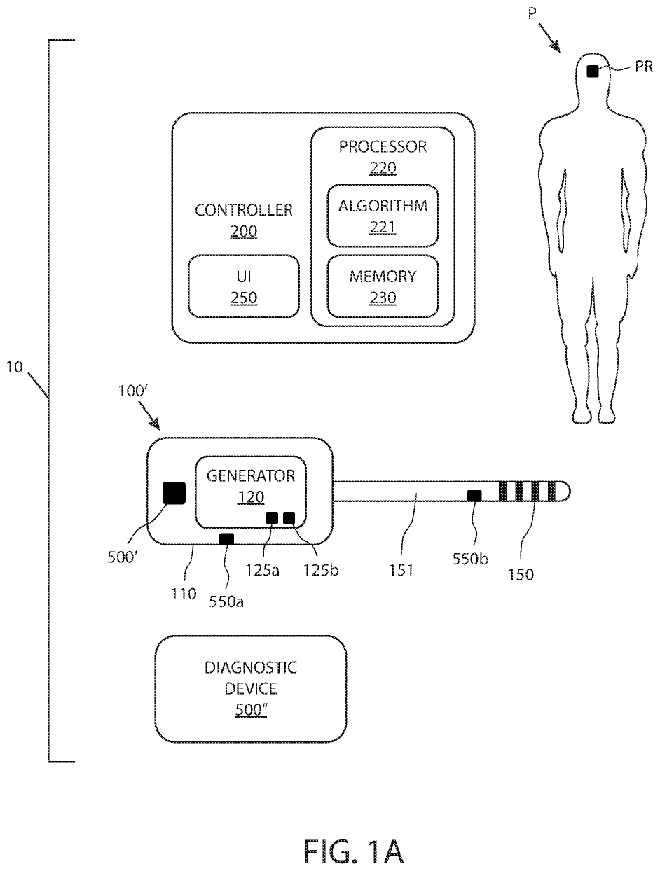

Referring now to FIG. 1, a schematic view of a system for delivering stimulation to a patient is illustrated, consistent with the present inventive concepts. System 10 comprises stimulator 100, controller 200 and diagnostic device 500. Stimulator 100 can be configured to deliver energy or other forms of stimulation (as described herein), such as via one or more stimulation elements (stimulation elements 150 described herebelow). System 10 can be used to treat one or more patient diseases or disorders of a patient P, such as when system 10 is configured to treat a disease and/or disorder selected from the group consisting of: a neurological disease; a neurological disorder; a psychiatric condition; Alzheimer's Disease (AD) such as Mild or Moderate Alzheimer's Disease; probable Alzheimer's Disease; a genetic form of Alzheimer's Disease; Mild Cognitive Impairment (MCI); hippocampal damage such as hippocampal damage due to Alzheimer's Disease, anoxia, epilepsy or depression; neuronal loss; neuronal damage; chemotherapy induced memory impairment; epilepsy; a seizure disorder; a movement disorder; essential tremor; dementia; amnesia; a memory disorder such a spatial memory disorder; head injury; traumatic brain injury; stroke; cognitive impairment associated with Schizophrenia; Parkinson's Disease related cognitive impairment or dementia; Parkinson-Plus syndrome; depression such as major depression; obsessive compulsive disorder (OCD); dystonia; addiction; cluster headache; anorexia nervosa; bipolar disorder; pain such as chronic pain; dementia such as dementia with Lewy bodies (DLB); Huntington's disease; obesity; Tourette syndrome; multiple sclerosis (MS) tremor; urinary function; and combinations of one or more of these. In some embodiments, system 10 is configured to treat two or more of the diseases and/or disorders listed above.

In some embodiments, system 10 is configured to treat Parkinson's Disease, such as when stimulation elements 150 are positioned to stimulate or otherwise affect a patient site selected from the group consisting of: Motor Thalamus; subthalamic area and nucleus; globus pallidus; subtantia nigra; pedunculopontine nucleus; and combinations of one or more of these. In some embodiments, system 10 is configured to treat depression, such as when stimulation elements 150 are positioned to stimulate or otherwise affect: Orbital Frontal Cortex; Prefrontal Cortex; Dorsolateral Prefrontal Cortex. Area 25; Subcallosal Cingulate; Medial Forebrain Bundle; Anterior Limb of Internal capsule; habenula; Inferior thalamic peduncle (ITP); and combinations of one or more of these. In some embodiments, system 10 is configured to treat essential tremor, such as when stimulation elements 150 are positioned to stimulate or otherwise affect: Cerebellar Surface; Cerebellar Peduncles; Motor Thalamus; Subthalamic area and nucleus; Motor cortex; and combinations of one or more of these. In some embodiments, system 10 is configured to treat or otherwise affect: Motor Cortex (somatotopically); PMC; SMA; Motor Thalamus; Pallidum; Brodmann Area 6; Brodmann Area 40/7 Globus pallidus; motor thalamus; subthalamic area or nucleus; and combinations of one or more of these. In some embodiments, system 10 is configured to treat essential Alzheimer's Disease and/or Mild Cognitive Impairment, such as when stimulation elements 150 are positioned to stimulate or otherwise affect: Fornix; mammillary bodies; nucleus basalis of Meynert; thalamus; Subcallosal Cingulate; Medial Forebrain Bundle; Anterior Limb of Internal apsule; habenula; Inferior thalamic peduncle (ITP); and combinations of one or more of these.

In some embodiments, system 10 is configured to treat a medical condition listed in the far-left column of Table A herebelow. In these embodiments, system 10 can be configured to stimulate one or more of the anatomical locations listed in the middle column of Table A, for each associated medical condition. Alternatively or additionally, system 10 (e.g. diagnostic device 500) can be configured to collect data related to a physiologic parameter of an anatomical location listed in the far right column of Table A, for each associated medical condition. In these embodiments, system 10 can collect physiologic data from the site of stimulation and/or another site (e.g. one or more of the sites listed in the far-right column).

TABLE A

| Medical Condition | | Example Physiologic |
|---|---|---|
| Treated | Example Stimulation Site | Parameter Measured |
| Parkinson's Disease | Motor Thalamus; subthalamic | Desired activity at: the Site |
| | area and nucleus; globus pallidus; | of Stimulation and/or Motor |
| | subtantia nigra; and/or | cortex; supplementary Motor |
| | pedunculopontine nucleus | cortex (SMA); premotor |
| | | cortex (PMC); cerebellum; |
| | | and/or cortical speech areas. |
| Depression and/or Mood | Orbital Frontal Cortex; Prefrontal | Desired activity at: the Site |
| Disorder | Cortex; Dorsolateral Prefrontal | of Stimulation; orbital frontal |
| | Cortex. Area 25; Subcallosal | cortex; Brodman area 6, 9 |
| | Cingulate; Medial Forebrain | 10, and/or 46; cingulate |
| | Bundle; Anterior Limb of Internal | gyrus; brainstem amygdala; |
| | capsule; habenula; and/or Inferior | cerebellum; hypothalamus; |
| | thalamic peduncle (ITP) | and/or hippocampus |
| Cerebellar Tremor; | Cerebellar Surface; Cerebellar | Desired activity at: the Site |
| Holmes Tremor; Tremor | Peduncles; Motor Thalamus; | of Stimulation; Motor cortex; |
| with Multiple Sclerosis; | Subthalamic area and nucleus; | supplementary Motor cortex; |
| Post Traumatic Tremor; | and/or Motor cortex. | premotor cortex cerebellum; |
| Post-Stroke Tremor; | | and/or cortical speech areas. |
| Dystonic tremor and/or | | |
| Essential Tremor | | |
| Dystonia such as Focal | Motor Cortex (somatotopically); | Desired activity at: the Site |
| Dystonia | PMC; SMA; Motor Thalamus; | of Stimulation; Motor cortex; |
| | Pallidum; Brodmann Area 6; | supplementary Motor cortex; |
| | Brodmann Area 40/7 Globus | cerebellum; and/or cortical |
| | pallidus; motor thalamus; and/or | speech areas |
| | subthalamic area or nucleus | |
| Alzheimer's Disease | Fornix; mammillary bodies; | Desired activity at: the Site |
| and/or Mild Cognitive | nucleus basalis of Meynert; | of Stimulation; Temporal |
| Impairment | thalamus; Subcallosal Cingulate; | cortex; hippocampus; |
| | Medial Forebrain Bundle; | parietal cortex; frontal |
| | Anterior Limb of Internal apsule; | cortex; and/or cerebellum |
| | habenula; and/or Inferior thalamic | |
| | peduncle (ITP) | |
| Auditory Hallucinations | Auditory Pathways; Cortex | Desired activity at: the Site |
| and/or Schizophrenia | Subcallosal Cingulate; Medial | of Stimulation; auditory |
| | Forebrain Bundle; Anterior Limb | pathways; and/or auditory, |
| | of Internal capsule; habenula; | language and/or speech |
| | and/or Inferior thalamic | cortex |
| | peduncle (ITP) | |
| Epilepsy | Seizure Focus (e.g. as identified | Desired activity at: the Site |
| | with EEG, PET, and/or SPECT). | of Stimulation; |
| | Fields of forel; and/or thalamus | hippocampus; amygdala; |
| | | and/or neocortex |
| Tourette's and Obsessive | Cortical Surface; Premotor Cortex | Desired activity at: the Site |
| Compulsive disorder | (PMC); Supplementary Motor | of Stimulation; Motor cortex; |
| | Cortex; Dorsolateral Prefrontal | supplementary Motor cortex; |
| | Cortex; Globus pallidus; | premotor cortex; cerebellum; |
| | Thalamus; Orbital Frontal Cortex; | and/or cortical speech and/or |
| | Prefrontal Cortex; Area 25; | language areas. |
| | Subcallosal Cingulate; Medial | |
| | Forebrain Bundle; Anterior Limb | |
| | of Internal capsule; and/or | |
| | habenula | |
| Stuttering | Cortical Surface; Motor Cortex; | Desired activity at: the Site |
| | Premotor Area; Motor Thalamus; | of Stimulation; speech and |
| | and/or Pallidum | language areas; motor |
| | | cortical areas; and/or |
| | | Brodman area 9, 10, and/or |
| | | 46 |
| Writer's Cramp | Motor Cortex (somatotopically); | Desired activity at: the Site |
| | PMC; SMA; Motor Thalamus; | of Stimulation; Motor cortex; |
| | and/or Pallidum | supplementary Motor cortex; |
| | | premotor cortex; cerebellum; |
| | | and/or cortical speech areas. |
| Dystonia such as Focal | Motor Cortex (somatotopically); | Desired activity at: the Site |
| Dystonia | PMC; SMA; Motor Thalamus; | of Stimulation; Motor cortex; |
| | Pallidum; Brodmann Area 6; | supplementary Motor cortex; |
| | Brodmann Area 40/7; Globus | premotor cortex; cerebellum; |
| | pallidus; motor thalamus; and/or | and/or cortical speech areas |
| | subthalamic area or nucleus | |
| Generalized Dystonia | Motor Cortex (somatotopically); | Desired activity at: the Site |
| | PMC; SMA; Motor Thalamus; | of Stimulation; Motor cortex; |
| | Pallidum; Brodmann Area 6; | supplementary Motor cortex; |
| | Brodmann Area 40/7; Globus | premotor cortex; cerebellum; |
| | pallidus; motor thalamus; and/or | and/or cortical speech areas. |
| | subthalamic area or nucleus | |
| Myoclonus | Motor Cortex (somatotopically); | Desired activity at: the Site |
| | PMC; SMA; Motor Thalamus; | of Stimulation; Motor cortex; |
| | Pallidum; Brodmann Area 6; | supplementary Motor cortex; |
| | Brodmann Area 40/7; Globus | premotor cortex; cerebellum; |
| | pallidus; and/or subthalamic area | and/or cortical speech areas. |
| | or nucleus | |
| Chorea/hemibalism | Motor Cortex (somatotopically); | Desired activity at: the Site |
| | PMC; SMA; Motor Thalamus; | of Stimulation and/or Motor |
| | Pallidum; Brodmann Area 6; | cortex; supplementary Motor |
| | and/or Brodmann Area 40/7 | cortex; premotor cortex; |
| | | cerebellum; cortical speech |
| | | areas. |
| Attention Deficit Disorder | Frontal Cortical Surface; | Desired activity at: the Site |
| and/or Hyperactivity | Prefrontal Cortical Surface | of Stimulation |
| Disorder | Orbital Frontal Cortex; Prefrontal | |
| | Cortex; and/or Dorsolateral | |
| | Prefrontal Cortex. Area 25; | |
| | Subcallosal Cingulate; Medial | |
| | Forebrain Bundle; Anterior Limb | |
| | of Internal capsule; habenula; | |
| | amygdala; nucleus accumbens; | |
| | and/or hippocampus | |
| Drug Dependence; | Cortical and/or Subcortical | Desired activity at: the Site |
| Alcohol Dependence; | Projections to Nucleus | of Stimulation; orbital frontal |
| Nicotine Dependence; | Accumbens; Nucleus Accumbens | cortex; Brodman area 6, 9 |
| Cocaine Dependence; | per se; Ventral Tegmental Area; | 10, and/or 46; cingulate |
| Dependence on Opiates; | and/or Medial Forebrain Bundle. | gyms; brainstem amygdala; |
| Dependence on | | cerebellum; hypothalamus; |
| Stimulants | | and/or hippocampus |
| Eating Disorder and/or | Cortical and/or Subcortical | Desired activity at: the Site |
| Obesity | Projections to Nucleus | of Stimulation; orbital frontal |
| | Accumbens; Nucleus Accumbens | cortex; Brodman area 6, 9 |
| | per se; Ventral Tegmental Area; | 10, and/or 46; cingulate |
| | Hypothalamus. Orbital frontal | gyms; brainstem amygdala; |
| | cortex; medial forebrain bundle; | cerebellum; hypothalamus; |
| | Orbital Frontal Cortex; Prefrontal | and/or hippocampus |
| | Cortex; and/or Dorsolateral | |
| | Prefrontal Cortex. Area 25; | |
| | Subcallosal Cingulate; Medial | |
| | Forebrain Bundle; Anterior | |
| | Limmb of Internal capsule; and/or | |
| | habenula | |
| Pain (deafferentation | Cingulate Gyrus; Insula; | Desired activity at: the Site |
| and/or nociceptive) | Secondary Somatosensory Cortex; | of stimulation |
| | Motor Cortex; thalamus; internal | |
| | capsule; medial lemniscus Orbital | |
| | Frontal Cortex; Prefrontal Cortex; | |
| | and/or Dorsolateral Prefrontal | |
| | Cortex. Area 25; Subcallosal | |
| | Cingulate; Medial Forebrain | |
| | Bundle; Anterior Limb of Internal | |
| | capsule; and/or habenula | |

Stimulator 100 can be positioned to deliver stimulation to one or more anatomical locations on and/or within patient P. System 10 can be configured to optimize (as described herein) delivery of stimulation by stimulator 100 in one or more ways. For example, system 10 can be configured to optimize delivery of stimulation by performing a function selected from the group consisting of: determine if stimulator 100 is delivering stimulation to a desired (e.g. pre-determined) anatomical location; determine if stimulator 100 should deliver stimulation to a second anatomical location versus a first anatomical location; determine if stimulator 100 should deliver stimulation to multiple anatomical locations (e.g. simultaneously or sequentially); determine if a selected (e.g. pre-determined) anatomical location is achieving a desired effect (e.g. a desired physiologic response); determine if stimulator 100 is causing an undesired effect to a non-target anatomical location; determine if stimulator 100 is delivering adequate stimulation (e.g. an adequate level of: stimulation intensity such as adequate energy, power, current and/or voltage; frequency of stimulation; concentration of a stimulating agent being delivered, and/or flow rate of a stimulation agent being delivered); determine if stimulator 100 is delivering optimized stimulation (e.g. optimized energy, power, current, voltage, frequency, concentration of an agent, and/or flow rate of an agent); determine if stimulator 100 is delivering stimulation that is at an undesired level (e.g. energy, power, current, voltage, frequency, concentration of an agent, and/or flow rate of an agent is above a safety level or otherwise at an undesired level); and combinations of one or more of these.

System 10 can be configured to assess one or more stimulation settings in a manual mode (e.g. performed by a clinician), a semi-automatic mode (e.g. performed partially by a clinician and partially by one or more components of system 10), and/or an automatic mode (performed fully by system 10 without significant clinician input). System 10 can be configured to adjust one or more stimulation settings in a manual, semi-closed loop and/or closed loop fashion. In some embodiments, system 10 is configured to adjust stimulation in an automatic and/or semi-automatic mode, with required confirmation of at least one adjustment by a user (e.g. a clinician confirming via a user interface of controller 200).

Stimulator 100 comprises housing 110 which surrounds generator 120. Stimulator 100 can comprise an implantable stimulator, an external stimulator (external to patient P), or a multi-component device in which one or more portions are implanted in patient P, and one or more portions are external to patient P. Stimulator 100 comprises one or more components configured to deliver stimulation, stimulation elements 150 (four shown in FIG. 1). Stimulator 100 can comprise a lead, lead 151 shown, which can extend from housing 110. One or more stimulation elements 150 can be positioned on lead 151. Alternatively or additionally, one or more stimulation elements 150 can be positioned on housing 110 or another component of stimulator 100. In some embodiments, stimulator 100 comprises at least a portion of diagnostic device 500, such as is described herebelow in reference to FIG. 1A. In some embodiments, stimulator 100 comprises one or more sensors, such as is described herebelow in reference to FIG. 1A.

Housing 110 can comprise one or more flexible or rigid materials, such as a material selected from the group consisting of: plastic; metal; titanium; stainless steel; and combinations of one or more of these. Housing 110 can comprise two housings that are sealed together, such as via adhesive, a weld, or the like. In some embodiments, housing 110 comprises two or more discrete housings, which may be connected by a conduit (e.g. a wire, tube and/or optical fiber) or unattached.

Generator 120 can comprise an electronic, mechanical, and/or other assembly configured to provide stimulation to the one or more stimulation elements 150 (e.g. via one or more wires, fluid delivery tubes, optical fibers, wave guides, and/or other energy transmitting conduits not shown). In some embodiments, generator 120 provides stimulation energy to one or more stimulation elements 150, such as energy selected from the group consisting of: electrical energy; microwave energy; magnetic energy; sound energy such as ultrasound energy; light energy; thermal energy; heat energy; cryogenic energy; chemical energy; and combinations of one or more of these. Alternatively or additionally, generator 120 can be configured to provide a pharmaceutical drug or other agent to one or more stimulation elements 150, such as when generator 120 comprises a pump or other fluid propulsion mechanism.

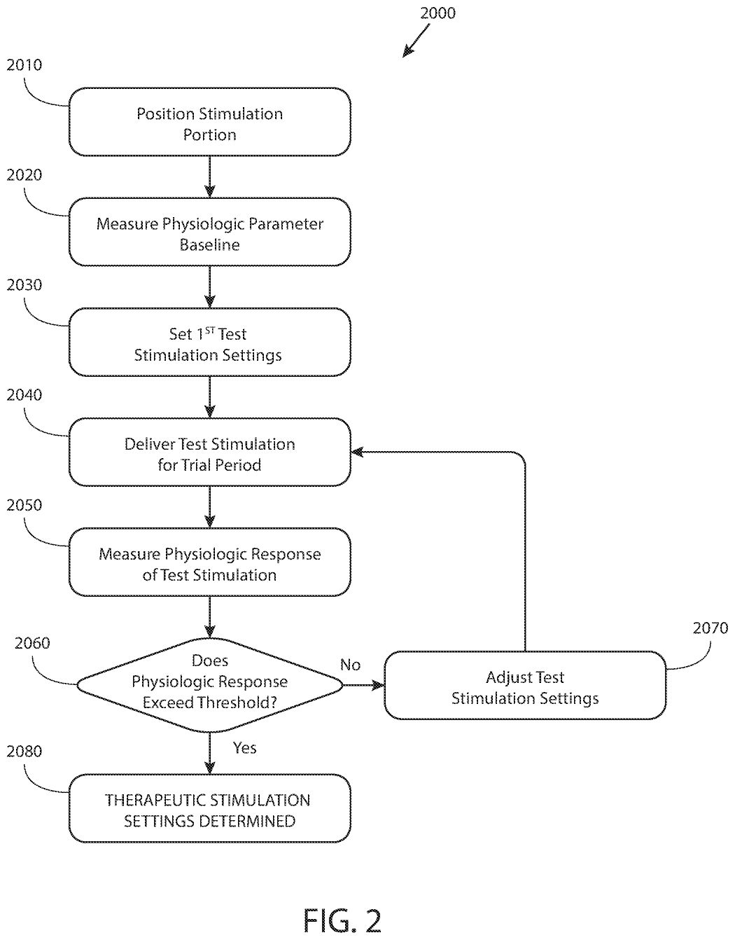

Stimulation elements 150 can comprise one or more elements configured to deliver energy, such as is described hereabove in reference to generator 120. In some embodiments, stimulation elements 150 comprises one or more electrodes configured to deliver electromagnetic energy to tissue. Alternatively or additionally, stimulation elements 150 can comprise an optical component, such as a lens or prism configured to deliver light energy to tissue. Alternatively or additionally, stimulation elements 150 can comprise a piezo material, such as an element to deliver sound energy to tissue, such as ultrasound energy delivered to tissue. Alternatively or additionally, stimulation elements 150 can comprise a drug delivery element, such as a needle, a fluid jet, an opening (e.g. an opening in a lead 151 configured as a drug delivery catheter), and/or an iontophoretic fluid delivery element, which can deliver one or more drugs or other agents delivered from generator 120 (e.g. when generator 120 comprises a reservoir, for example a refillable reservoir not shown). Stimulation elements 150 can be positioned (e.g. implanted and/or otherwise placed) proximate one or more Patient P tissue locations, such as is described herebelow in reference to FIG. 2. In some embodiments, stimulation elements 150 comprise one or more elements that deliver (e.g. singly or collectively deliver) both energy and an agent (e.g. a pharmaceutical agent). For example, a single stimulation element 150 can deliver both energy and an agent, or a first stimulation element 150 can deliver energy and a second stimulation element 150 can deliver an agent.

In some embodiments, one or more stimulation elements 150 comprise a sensor, such as an electrode configured to record electrical activity of the patient (e.g. electrical activity in tissue proximate the sensor), or other sensor, such as is described herebelow in reference to sensor 550 of FIG. 1A. In these embodiments, the electrode can also be configured to deliver electromagnetic energy to tissue (e.g. the stimulation energy).

Controller 200 can comprise a user interface, UI 250, which can comprise one or more user input and/or user output components such as: keyboard; mouse; keypad; switch; membrane switch; touchscreen; display; audio transducer such as a speaker or buzzer; vibrational transducer; light such as an LED; and combinations of one or more of these. Controller 200 can comprise a microcontroller, microprocessor, or other processing unit, processor 220. Processor 220 can comprise volatile or non-volatile electronic memory circuitry, memory 230. Processor 220 can further comprise one or more algorithms, algorithm 221. In some embodiments, diagnostic device 500 comprises at least a portion of algorithm 221 (e.g. diagnostic device 500 comprises at least a portion of processor 220 and/or controller 200). In some embodiments, algorithm 221 is configured to assess a measured physiologic parameter of patient P, such as to determine and/or otherwise assess a physiologic response of the patient to stimulation (e.g. test stimulation) as described herein. Alternatively or additionally, algorithm 221 can be configured to adjust one or more stimulation settings, also as described herein. In some embodiments, memory 230 stores information related to a desired physiologic response, such that a measured physiologic response can be compared to the desired physiologic response semi-automatically or automatically (“automatically” herein) via algorithm 221. In a non-limiting example, a desired “signature” of neural activity in tissue (e.g. in brain tissue), can be compared to a measured level of neural activity, and stimulation settings can be adjusted (e.g. via algorithm 221) to cause the measured level to approximate the desired level. In some embodiments, memory 230 stores one or more desired signatures of neural activity in tissue. System 10 can be configured to store one or more desired levels of one or more physiologic parameters and/or one or more desired physiologic responses (e.g. changes in physiologic levels), such as to be compared to a measured level of either. In some embodiments, algorithm 221 is configured as described herebelow.

Stimulator 100 is configured to deliver stimulation based on a set of stimulation settings 125. Stimulation settings 125 can comprise one or more stimulation settings selected from the group consisting of: anatomical position of one or more stimulation elements 150, anatomical position of lead 151, selection of a subset of stimulation elements 150 (e.g. selection of one or more stimulation elements 150 from a set of two or more stimulation elements 150); amplitude of energy delivery (e.g. amplitude of current, voltage and/or sound level delivered); frequency of stimulation delivered (e.g. frequency of electrical energy, sound energy and/or light energy); pulse width of energy delivery (e.g. on time versus off time in continuous or intermittent modes); duration of stimulation delivery (e.g. number of hours per day in intermittent modes); time period in which stimulation is not to be delivered (e.g. avoidance of energy delivery during a period of activity such as driving and/or avoidance of energy delivery during a period of non-activity such as sleep); flow rate of delivery of an agent; concentration of an agent being delivered; pressure at which an agent is delivered; duration of stimulation; and combinations of one or more of these.

Stimulator 100 is configured to deliver stimulation in at least two forms: stimulation used to assess a physiologic response, “test stimulation”, and stimulation used to treat one or more patient diseases or disorders, “therapeutic stimulation”. Generator 120 can be configured to deliver the test stimulation based on one or more sets of stimulation settings, test stimulation settings 125a. In some embodiments, test stimulation settings 125a are adjusted based on an assessment of one or more physiologic parameters, such as is described herein. In some embodiments, test stimulation settings 125a are adjusted (e.g. automatically or semi-automatically) by algorithm 221 of controller 200.

Diagnostic device 500 can comprise an imaging device or other device configured to assess a physiologic response of patient P, response PR. Diagnostic device 500 can comprise one or more devices, configured to measure one or more physiologic parameters of the patient. The one or more physiologic parameters measured can be used as surrogate markers for safety, efficacy, and/or other performance level of the stimulation provided by stimulator 100. In some embodiments, diagnostic device 500 measures at least one surrogate physiologic parameter representative of achievement of therapeutic benefit, and at least one surrogate physiologic parameter representative of occurrence of an undesired event. Diagnostic device 500 can comprise an external device and/or an implantable device, or a device that includes both external and implantable portions. Diagnostic device 500 can comprise one, two or more devices selected from the group consisting of: magnetic resonance imaging (MRI) such as functional magnetic resonance imaging (fMRI); electroencephalograph (EEG); magnetoencephalograph (MEG); positron emission tomography (PET) scanner; local field potential (LFP) recording device; neuronal spike recording device; MR spectroscopy device; MR angiography device; a regional blood flow measurement device (e.g. a device using perfusion CT and/or stable xenon CT); and combinations of one or more of these. In some embodiments, diagnostic device 500 comprises at least two of: magnetic resonance imaging (MRI) such as functional magnetic resonance imaging (fMRI); electroencephalograph (EEG); magnetoencephalograph (MEG); or positron emission tomography (PET) scanner; local field potential (LFP) recording device; neuronal spike recording device; MR spectroscopy device; MR angiography device; or a regional blood flow measurement device (e.g. a device using perfusion CT and/or stable xenon CT). In some embodiments, at least a portion of diagnostic device 500 is positioned in stimulator 100, such as when stimulator 100 comprises an implantable and/or external portion that comprises diagnostic device 500. For example, stimulator 100 can comprise one or more electrodes used to record neuronal activity, such as a recording including: neuronal spikes, LFP activity and/or EEG activity. In some embodiments, diagnostic device 500 comprises at least two devices used to measure the same or different physiologic parameters and/or the same or different physiologic responses of the patient. Examples of physiologic parameters that are measured, quantified, qualified, compared (e.g. to a previous level to determine a physiologic response) and/or otherwise assessed by diagnostic device 500 and/or another component of system 10 include but are not limited to: neuronal firing activity (e.g. a quantification of the number of neurons firing in a volume of tissue); neuronal firing rate; power of brain activity; rhythm of brain activity; phase-amplitude coupling of brain activity; cerebral vascular transit time; glucose handling; and combinations of one or more of these. In some embodiments, a measured physiologic parameter is derived from a magnetoencephalography signal generated by the brain at rest and after stimulation using a set of test stimulation settings 125a. In some embodiments, a measured physiologic parameter is derived from a PET scan signal generated by the brain at rest and after stimulation using a set of test stimulation settings 125a. In some embodiments, a measured physiologic parameter is derived from an fMRI signal generated with the brain at rest and after stimulation using a set of test stimulation settings 125a. In use of these various diagnostic devices 500, stimulation can be applied to one or more of: the deep brain, the brain cortex and/or the brain sub-cortex.

In some embodiments, diagnostic device 500 comprises one, two, or more sensors, such as sensor 550 described herebelow in reference to FIG. 1A. In some embodiments, diagnostic device 500 provides diagnostic information (e.g. measured physiologic parameter information) directly to controller 200 (e.g. in a feedback loop) via connection 510 shown. Connection 510 can comprise a wired connection and/or a wireless connection (e.g. via Bluetooth or other communication elements included in controller 200 and diagnostic device 500). Connection 510 can provide semi-automatic and/or fully automatic adjustment (e.g. closed loop adjustment) of stimulation (e.g. test stimulation) provided by stimulator 100, the adjustment based on the information collected by diagnostic device 500. In some embodiments, closed loop adjustment of stimulation requires configuration by a clinician prior to stimulation delivery. Closed loop adjustment of stimulation can be performed within limits (e.g. predetermined maximums and/or minimums of stimulation parameters), such as limits set by a clinician prior to use. The feedback loop can be configured to reduce programming time and/or simplify programming.

System 10 can be configured to measure the physiologic parameters after the test stimulation has been delivered for a duration of time, a “test duration” TD. Test duration TD can comprise a minimum period of time and/or a target period of time, such as is described herebelow in reference to FIG. 2. In some embodiments, a first test stimulation that is performed for a first test duration T_{D} at a first set of test stimulation settings 125a′ is followed by a second test stimulation that is performed for a second test duration TD₂ at a second set of test stimulation settings 125a″. In these embodiments, the second test stimulation can be performed after a particular “waiting duration” WD has elapsed (e.g. elapsed since the first test stimulation has completed), such as a waiting duration WD comprising a minimum or maximum time period, also as described herebelow in reference to FIG. 2. The first and second test durations TD₁ and TD₂ can comprise similar or dissimilar time periods. In some embodiments, three or more test stimulations can be performed, with similar and/or dissimilar test durations TD and waiting durations WD.

In some embodiments, stimulator 100 delivers stimulation comprising both delivery of energy and delivery of an agent. In these embodiments, optimization of stimulation can comprise optimization of energy delivery, optimization of agent delivery, or both. Algorithm 221 can be configured (e.g. using data collected by diagnostic device 500) to optimize the energy delivery, optimize the agent delivery, or both.

In some embodiments, algorithm 221 is configured to perform at least two assessments. For example, algorithm 221 can be configured to both analyze one or more physiologic parameters to assess therapeutic benefit, and to analyze one or more similar and/or dissimilar physiologic parameters to assess presence of an adverse event or other undesired event (e.g. presence of an undesired response to stimulation).

In some embodiments, algorithm 221 is configured to compare two or more of the following: delivery of stimulation to a first location; delivery of stimulation to a second location; and/or delivery of stimulation to both the first location and the second location. For example, system 10 can be configured to compare all three of these and deliver stimulation based on the result of the comparison.

Algorithm 221 can comprise one or more biases. In some embodiments, algorithm 221 comprises a bias that preferentially avoids stimulation settings 125 that result in an adverse event and/or other undesired event (e.g. when also attempting to optimize therapeutic benefit). For example, algorithm 221 can assess two sets of stimulation settings 125 that provide similar therapeutic benefit, making the selection via a bias that selects the set of stimulation settings 125 that has reduced undesired events (e.g. reduced undesired side effects). In some embodiments, algorithm 221 comprises a bias related to a non-therapeutic parameter, such as stimulator 100 battery life. For example, if two sets of stimulation settings 125 provide similar therapeutic benefit, algorithm 221 can include a bias that selects the set of stimulation settings 125 that correlate to another (non-therapeutic) goal, such as by selecting stimulation settings 125 that require less energy delivered over time (e.g. to prolong the battery life of stimulator 100). Other examples of biases of algorithm 221 include, but are not limited to, biases that are related to: desired versus undesired implantation locations of stimulation elements 150 and/or other portion of stimulator 100 (e.g. biased to reduce surgical complexity or long-term implantation issues); desired versus undesired system configurations (e.g. biased to achieve a simpler system such as a bias toward less stimulation elements 150 and/or less leads 151); and/or desired versus undesired agent to be delivered (e.g. biased to achieve favorable cost, health insurance coverage, availability, and/or side effects of the agent).

In some embodiments, stimulator 100 comprises all or a portion of algorithm 221 (e.g. stimulator 100 comprises all or a portion of controller 200). In some embodiments, stimulator 100 comprises a first portion of algorithm 221 and controller 200 comprises a second portion of algorithm 221.

Referring now to FIG. 1A, a schematic view of a system for delivering stimulation to a patient is illustrated, the system comprising a stimulator that includes an integral diagnostic device, consistent with the present inventive concepts. System 10 comprises stimulator 100′ and controller 200. Stimulator 100′ can comprise a diagnostic device of the present inventive concepts, diagnostic device 500′ which can be integral to stimulator 100 (e.g. contained within one or more housings of stimulator 100, such as housing 110). In some embodiments, system 10 further comprises another diagnostic device, such as diagnostic device 500″ shown, for example an external diagnostic device. System 10, stimulator 100′, controller 200, diagnostic device 500′ and/or diagnostic device 500″ can be of similar construction and arrangement to the similar devices described hereabove in reference to FIG. 1.

In some embodiments, stimulator 100′ comprises an implantable stimulator (e.g. an implantable brain stimulator or an implantable pain treatment stimulator), and diagnostic device 500′ comprises a diagnostic device configured to measure and/or assess electrical signals received from one or more electrode-based stimulation elements, such as electrical signals representing: neuronal spikes, LFP activity and/or EEG activity. In these embodiments, stimulator 100′ can be configured to adjust (e.g. automatically adjust) stimulation settings 125 (e.g. test stimulation settings 125a and/or therapeutic stimulation settings 125b described hereabove in reference to FIG. 1) in a closed loop fashion based on measurements of one or more physiologic parameters made by the diagnostic device 500′ portion of stimulator 100′. These adjustments to stimulation settings 125 can be performed with and/or without the use of a separate diagnostic device 500″ (e.g. an external diagnostic device). These adjustments to stimulation settings 125 can be performed on a relatively continual and/or intermittent basis, such as when closed loop adjustments to stimulation settings 125 are performed: at least once a day; at least once a week; at least once a month; and/or at least once every 3 months.

In some embodiments, diagnostic device 500′ and/or 500″ is configured to measure a physiologic parameter selected from the group consisting of: a brain chemical; a neurotransmitter; a protein; pH; glucose; and combinations of one or more of these. In some embodiments, diagnostic device 500′ or another portion of stimulator 100′ comprises one, two or more sensors 550, such as implantable sensors 550a and/or 550b shown (sensor 550a positioned on housing 110, sensor 550b positioned on lead 151). Sensor 550a and/or 550b can be attached to electronic or other sensor-interfacing circuitry of diagnostic device 500′ via one or more wires or other information transmitting conduits (conduits and/or circuitry not shown). Sensor 550a and/or 550b can be configured to measure one or more physiologic parameters of the patient. Sensor 550a and/or 550b can each comprise one or more sensors selected from the group consisting of: a sensor configured to measure electrical activity such as a sensor comprising an electrode (e.g. an electrode-based stimulation element 150); a chemical sensor; an electromagnetic sensor; a pressure sensor; a temperature sensor; a neurotransmitter sensor; a protein sensor; a pH sensor; a glucose sensor; and combinations of one or more of these. In some embodiments, system 10 is configured to adjust one or more stimulation settings 125 based on a diagnostic device 500 (e.g. diagnostic device 500′ and/or 500″) measuring at least two physiologic parameters of the patient (e.g. when determination that a desired physiologic response has been achieved is based on the measurement of the at least two physiologic parameters).

In some embodiments, diagnostic device 500′ provides diagnostic information (e.g. measured physiologic parameter information) directly to controller 200 (e.g. in a feedback loop), such as is described hereabove in reference to connection 510 of FIG. 1.

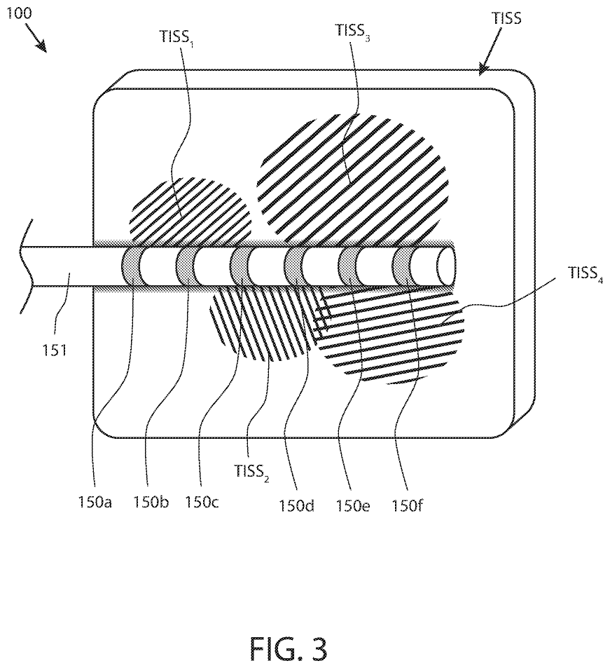

Referring now to FIG. 2, a method of delivering a stimulation treatment to a patient is illustrated, consistent with the present inventive concepts. Referring additionally to FIG. 3, a schematic view of a stimulation portion of a stimulator positioned proximate multiple anatomical locations is illustrated, consistent with the present inventive concepts. Lead 151, including multiple stimulation elements 150 has been inserted into a volume of tissue, tissue volume TISS shown. While the stimulation elements 150 are shown in a linear (one dimensional) orientation, it should be understood that stimulation elements 150 can be positioned in a two dimensional orientation (e.g. multiple locations in a single plane) or a three-dimensional orientation (e.g. multiple locations in multiple planes).

Method 2000 of FIG. 2, and stimulator 100 of FIG. 3 will be described using the components of system 10 described herein. Method 2000 can be performed to treat a disease or disorder of a patient, also as described herein. In Step 2010, at least a portion of stimulator 100 is positioned relative to the patient, such that one or more stimulation elements 150 can deliver stimulation to the patient (e.g. deliver stimulation energy and/or a stimulating agent). In some embodiments, at least a portion of stimulator 100 is implanted in the patient, such as when a stimulator 100 comprising lead 151 and including one, two or more stimulation elements 150 (e.g. electrodes) are implanted in the patient. In some embodiments, stimulation elements 150 are positioned proximate both target anatomical locations (e.g. anatomical locations desired to be stimulated) and non-target anatomical locations (e.g. anatomical locations in which no or minimal stimulation is desired). In some embodiments, stimulation elements 150 (e.g. multiple electrodes or other stimulation elements such as stimulation elements 150a-f shown) are positioned within a volume of tissue (tissue volume TISS shown). Stimulation elements 150a-f are positioned proximate multiple sub-portions of tissue volume TISS, tissue portions TISS₁, TISS₂, TISS₃, and/or TISS₄, shown in FIG. 3). In some embodiments, different effects are desired in one tissue portion versus another. TISS₁₋₄ can include a patient anatomical area selected from the group consisting of: a location on the skin; a skin location proximate an organ; a skin location proximate the brain; a skin location proximate the spine; an implant location (e.g. under the patient's skin); a location proximate the brain; a location proximate the vagus nerve; a location proximate the spine; a location proximate the heart; a location proximate the kidney; a location proximate the stomach; and combinations of one or more of these.

In some embodiments, one or more stimulation elements 150 are implanted or otherwise positioned proximate tissue selected from the group consisting of: brain tissue; deep brain tissue; cortical brain tissue; nerve tissue; vagus nerve tissue; organ tissue; heart tissue; kidney tissue; pancreatic tissue; spinal cord tissue; central nervous system tissue; peripheral nervous system tissue; dorsal root ganglia; and combinations of one or more of these.

In some embodiments, one or more stimulation elements 150 are positioned proximate brain tissue selected from the group consisting of: Papez Circuit; hippocampus; cingulate gyrus; fornix; a mammillothalamic tract; amygdala; hypothalamus; mammillary bodies; septal nuclei; temporal neocortex; the medial forebrain bundle (MFB); anterior and mediodorsal nuclei of the thalamus; the diagonal band of the Broca; temporal stem and temporal white matter; brainstem; nucleus basalis of Meynert; anterior thalamic nucleus; entorhinal cortex; rhinal cortex; periventricular zone; anterior thalamus; anterior insula; caudate; dorsal anterior cortex; dorsal cingulate; medial frontal cortex; nucleus accumbens; orbital frontal cortex; parietal region; periaqueductal gray area; posterior cingulate area; subcallosal area; subcallosal cingulate; subgenual cingulate; Brodmann area 10; Brodmann area 24; Brodmann area 25; Brodmann area 11/Brodmann area 10; Brodmann area 24b; Brodmann area 31; Brodmann area 32/Brodmann area 10; Brodmann area 32/Brodmann area 11; Brodmann area 39; Brodmann area 46; Brodmann area 46/Brodmann area 9; Brodmann area 47; Brodmann area 6; Brodmann area 9; one or more of Brodmann areas 1 through 52; ventral/medial prefrontal cortex area; ventral/medial white matter; dorsolateral prefrontal cortex; premotor cortex; ventrolateral prefrontal cortex; dorsal anterior cingulate caudate nucleus; frontal pole periaqueductal gray area; dorsolateral prefrontal area; subsingular cingulate; parahippocampal cortex; parahippocampal gyrus; ventral capsule; ventral striatum; ventral intermediate nucleus (VIM); ventral oralis posterior nucleus (VOP); ventral oralis anterior nucleus (VOA); posterior ventral globus pallidus internal; anterior medial globus pallidus internal; globus pallidis external; subthalamic nucleus; putamen; centromedian nucleus; zona incerta; preleminscal radiation; pedunculopontine nucleus; ventral posteromedial nucleus; ventral posterolateral nucleus; superior cingulum; septal area; ventral oral internal thalamic nuclei; dorsal medial nucleus; inferior thalamic peduncle; bed nucleus of the stria terminalis; stria terminalis; subcallosal cingulate gyrus CG25; latterly rostral anterior cingulate cortex CG24; lateral habenula; area LC of the caudate nucleus; anterior medial hypothalamus; posterior medial hypothalamus; lateral hypothalamus; orbito-frontal area; and combinations of one, two or more of these.

In some embodiments, one or more stimulation elements 150 are implanted proximate the brain of the patient to treat a neurological or psychological disorder of the patient. In some embodiments, one, two or more stimulation elements 150 are implanted proximate the spine of the patient to treat pain of the patient.

In Step 2020, diagnostic assembly 500 is used to measure one or more physiologic parameters of the patient, such as to record a baseline condition of one or more physiologic parameters of the patient, PR_{B} (e.g. a baseline of one or more physiologic parameters in TISSs₁₋₄). The baseline condition can comprise a state in which no stimulation has been given (e.g. within a time period of at least 30 seconds, 60 seconds, 5 minutes, 30 minutes, 1 hour, 1 day, 1 week or 1 month).

In Step 2030, stimulator 100 is configured to deliver a first test stimulation based on (e.g. using) a first set of test stimulation settings 125a′, such as a set including one or more stimulation settings 125 described hereabove in reference to FIG. 1.

In Step 2040, stimulator 100 delivers stimulation (e.g. an agent and/or stimulation energy) based on the first set of stimulation settings 125a′. Step 2040 can be performed for a pre-determined trial period, test duration TD (e.g. a minimum period of time or a target period of time), after which Step 2050 is performed. In some embodiments, the test duration TD comprises a minimum period of time, such as a minimum of 1 second, 2 seconds, 3 seconds, 5 seconds, 10 seconds, or 20 seconds. In some embodiments, test duration TD comprises a minimum time period of at least 1 minute, 5 minutes, 15 minutes, or 1 hour. In some embodiments, the test duration TD comprises a time period of no more than 5 minutes, no more than 15 minutes, no more than 1 hour, no more than 1 day, no more than 1 week, no more than 1 month or no more than 3 months. In some embodiments, test duration TD comprises a target period of time of 5 seconds, 10 seconds, or 20 seconds. In some embodiments, test duration TD comprises a target period of time of 1 minute, 5 minutes, 15 minutes, or 1 hour.

In some embodiments, diagnostic device 500 comprises an MRI, and test duration TD comprises a minimum period of at least 3 seconds. In some embodiments, diagnostic device 500 comprises an electroencephalograph, and test duration TD comprises a minimum period of at least 1 second. In some embodiments, diagnostic device 500 comprises an MRI, an electroencephalograph, or other diagnostic device as described herein, and test duration TD comprises a maximum period of 1 hour, or 2 hours.

In Step 2050, diagnostic assembly 500 is used to measure one or more physiologic parameters of the patient, parameters PR₁, such as to determine the physiologic response to the test stimulation applied in Step 2040.

In Step 2060, an assessment of parameters PR₁ is performed, such as to assess a physiologic response of the patient to the test stimulation. For example, and as shown in FIG. 2, if parameters PR₁ exceeds a threshold, step 2080 is performed, otherwise Step 2070 is performed. As used herein, exceeding a threshold shall include meeting a desired result (e.g. a desired efficacy of stimulation by stimulator 100), such as when a physiologic response is above a minimum threshold, below a maximum threshold, within a range of desired levels and/or outside of a range of undesired levels. The physiologic response can comprise a discrete level of a physiologic parameter, or a relative change in a physiologic parameter (e.g. a change that results when stimulation is applied and/or changed). In some embodiments, system 10 includes an algorithm, algorithm 221 described herein, which is configured to perform one or more assessments (e.g. parameter PR₁ assessments) of Step 2060. In some embodiments, algorithm 221 comprises a biased algorithm, also as described herein.

In Step 2070, the test stimulation settings 125a′ are adjusted, for example to a second set of test stimulation settings 125a″, and Step 2040 is performed again. In some embodiments, the repeat of Step 2040 is performed after a period of time, waiting duration WD (e.g. a period of time between the completion of Step 2040 or 2050, and the initiation of the adjusted stimulation performed in a repeat of Step 2040). Waiting duration WD can comprise a minimum period of time (e.g. to minimize prolonged effects of a previous stimulation), such as a time of at least 10 seconds, at least 30 seconds, at least 1 minute, at least 15 minutes, or at least 1 hour. Waiting duration WD can comprise a maximum period of time (such as to minimize other desired changes and/or to improve efficiency of the procedure), such as a time of no more than 1 minute; no more than 5 minutes; no more than 30 minutes; no more than 1 hour; or no more than 4 hours. In some embodiments, no stimulation is delivered by stimulator 100 during the waiting duration WD. Stimulation settings 125 can be adjusted using an algorithm, such as algorithm 221 described herein. Algorithm 221 can comprise one or more biases, such as a bias that preferentially selects a set of stimulation settings 125 that result in reducing an undesired event and/or achieving a non-therapeutic goal (e.g. prolonged battery life of stimulator 100).

In some embodiments, Step 2070 includes the adjustment of the selection of one or more stimulation elements 150 between a first test stimulation and a second test stimulation (e.g. one or more electrodes or other stimulation elements 150 is selected versus one or more different stimulation elements 150, such as to change a pattern of stimulation). Alternatively or additionally, one or more stimulations elements 150 can be repositioned, such as by adjusting the location of lead 151.

In some embodiments, Step 2070 includes an adjustment of a stimulation signal (e.g. to achieve a desired physiologic response in target or non-target anatomical locations). In some embodiments, an energy-based stimulation signal is adjusted by varying a signal property such as: intensity (e.g. voltage, current, amplitude, and/or magnetic field strength), frequency (e.g. variation of one or more frequencies of a signal); and/or pulse width. In some embodiments, an agent-based stimulation signal is adjusted by varying a single property such as: flow rate of agent being delivered; concentration of agent being delivered; and/or pressure at which agent is delivered.

In some embodiments, Step 2070 includes both an adjustment in the set of stimulation elements 150 delivering stimulation, and an adjustment in the stimulation signal. In these embodiments, the selection of the stimulation elements 150 can be performed (e.g. in one or more repeated loops of Steps 2040-2060 to determine a desired set of stimulation elements 150), after which adjustment of the stimulation signal can be performed (e.g. in one or more repeated loops of Steps 2040-2060 to determine a desired stimulation signal). In these embodiments, stimulation can comprise electrical stimulation, such as electrical stimulation provided by one, two or more electrode-based stimulation elements 150. The stimulation waveform provided by generator 120 and delivered by stimulation elements 150 can be adjusted in various ways, such as by varying one or more of: frequency of one or more waveforms of the stimulation (e.g. a frequency variation between 1 Hz and 10,000 Hz); pulse width of one or more pulses of one or more waveforms of the stimulation (e.g. a pulse width variation between 1 μsec and 500 μsec); intensity of one or more portions of one or more waveforms of the stimulation (e.g. a peak or average current variation between 0.1 mA and 10 mA and/or a peak or average voltage variation between 1.0V and 10.0V); a theta burst property (e.g. on or off); stimulation pattern (e.g. a variation in stimulation waveform shape); and combinations of one or more of these. For example, when stimulation elements 150 are positioned proximate a brain circuit with 4 nodes, TISS₁₋₄, it may be desirable for TISS₁ and TISS₃ to have increased activity, while activity decreases in TISS₂ and TISS₄. Stimulation provided by stimulator 100 can be adjusted, as described herein (e.g. via looping through Steps 2040 through 2060), to achieve those or other objectives (e.g. objectives defined prior to and/or during placement of stimulation elements 150).

In Step 2080, the therapeutic stimulation settings 125b of stimulator 100 are programmed based on the last set (the acceptable set) of test stimulation settings 125a (e.g. the therapeutic stimulation settings 125b equate to the last set of test stimulation settings 125a). Step 2080 can further include stimulator 100 providing the therapeutic stimulation using the newly set or otherwise established (“established” herein) therapeutic stimulation settings 125b. Therapeutic stimulation provided in Step 2080 can be delivered intermittently (e.g. varying by time of day, varying by a patient condition, and the like) and/or the therapeutic stimulation can be provided continuously. In some embodiments, the stimulation provided in Step 2080 is provided for at least 1 week, at least 1 month, at least 6 months, or at least 1 year. In some embodiments, the method 2000 of FIG. 2 is repeated, such as by returning to Step 2020 in which a new baseline of one or more physiologic parameters is determined and the remaining steps are performed, or by returning to Step 2030 (bypassing Step 2020) in which a new set of test stimulation settings 125a are established, and the remaining steps are performed (e.g. a subsequent test stimulation and measurement of a physiologic response as described herein). A repeated (e.g. second) performance of method 2000 can be performed at least 1 hour, at least 1 day, at least 1 week, at least 1 month, at least 6 months, or at least 1 year after a prior performance of method 2000.

In some embodiments, after performance of Step 2050 (an initial performance or a performance in a subsequent loop), it is desired to increase a response (e.g. neural activity) in one or more anatomical areas (e.g. TISS₁ and TISS₂) while decreasing activity in one or more different anatomical areas (e.g. TISS₃ and TISS₄). As a non-limiting example, one or more stimulation settings can be adjusted (e.g. in Step 2070) to achieve an increase in activity of 15% in TISS₁, while causing a decrease in activity of 10% in TISS₂, while causing an increase in activity of 5% in TISS₃. Stimulation (e.g. that achieves a desired physiologic response) of one or more of tissue volumes TISS₁₋₄, and/or avoidance of affecting one or more of these tissue volumes, can be decided based on previous studies that achieved a desired therapeutic outcome.

In some embodiments, diagnostic device 500 of the present inventive concepts is used to “target” a particular anatomical location (e.g. a brain network) and identify desired changes within that location that the stimulation provided by stimulator 100 needs to produce.

In some embodiments, system 10 is configured to operate in a relatively closed loop configuration in which diagnostic device 500 (e.g. an implantable diagnostic device 500 and/or an external diagnostic device 500) produces information (e.g. information regarding one or more physiologic parameters of the patient) and provides that information to a component of system 10 (e.g. controller 200 and algorithm 221) such as to cause a modified set of stimulation settings 125 to be delivered (e.g. in a test stimulation and/or therapeutic stimulation). In these embodiments, the effects of the modified stimulation can be re-measured (e.g. in a repeat of Step 2050), assessed (e.g. in a repeat of Step 2060), and continued in a loop until an optimized or otherwise desired therapeutic result is achieved.

Visualization of a physiologic response has been demonstrated in previous clinical studies. Stefurak et al (Movement Disorders, Volume 18, No. 12, 2013, 1508-1541) showed that a change in specific brain circuit activity (produced by deep brain stimulation) can be detected by fMRI. Davis et al (J Neurosurg, Volume 92, January 2000, 64-69) showed that activation of the anterior cingulate cortex (by thalamic stimulation) can be visualized by a PET scanner. Davis et al (Nature Medicine, Volume 3, June 1997, 671-674) showed that activation of the cortical motor system (by globus pallidus stimulation) can also be visualized by a PET scanner. Ray et al (Biomed Imaging Interv J, January 2007) showed that brain activity (during deep brain stimulation) can be visualized by magnetoencephalography (MEG). Laxton et al (Ann Neurol, Volume 68, 2010, 521-534) showed that a change in specific brain circuit activity (produced by deep brain stimulation) can be detected by EEG.

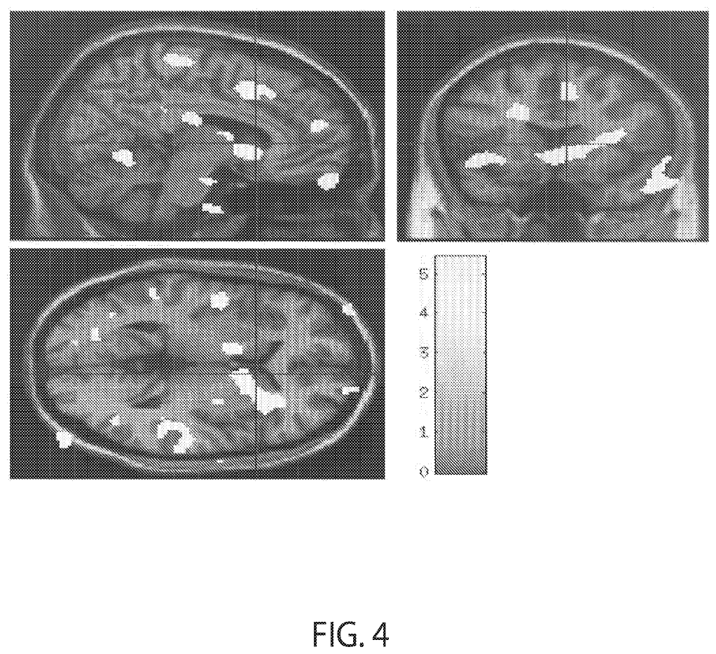

Applicant has performed studies utilizing the systems, methods and devices of the present inventive concepts. Data was successfully and safely collected with a diagnostic device 500 comprising an MRI operating 3 Tesla (article in Press JNS by Lozano). In some embodiments, diagnostic device 500 comprises an MRI operating at between 1.5 Tesla and 7 Tesla. Applicant has demonstrated that stimulator 100 can be safely turned on and off within an MRI environment (e.g. when diagnostic device 500 includes at least an MRI and one or more stimulation settings 125 are adjusted during operation of the MRI). System 10 can be configured to detect a change in specific brain circuit activity produced when stimulation elements 150 are positioned proximate deep brain tissue. Results of these studies are shown in FIG. 4 which illustrates a change in brain activity as visualized when diagnostic device 500 comprises an fMRI. Data illustrated in FIG. 4 is a comparison of baseline brain activity (i.e. no stimulation provided by stimulator 100) versus unilateral stimulation provided by stimulator 100 for approximately 10 seconds.

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

1. A stimulation system for treating a patient, comprising:
a stimulator comprising at least one stimulation element for delivering stimulation to the patient, the delivered stimulation based on a set of stimulation settings,
wherein the set of stimulation settings comprise a set of test stimulation settings and a set of therapeutic stimulation settings,
wherein the stimulation comprises test stimulation delivered to the patient for a test duration, and therapeutic stimulation delivered to the patient for an extended duration, and
wherein the test stimulation is based on the set of test stimulation settings and the therapeutic stimulation is based on the set of therapeutic stimulation settings;
a controller for adjusting a stimulation setting of the stimulator; and
a diagnostic device for measuring a physiologic response to the test stimulation on the patient;
wherein the set of therapeutic stimulation settings is based on an assessment of the measured physiologic response.

2.-139. (canceled)
